(19) United States
(12) Patent Application Publication
(10) Pub. No.: US 2010/0297017 A1
(43) Pub. Date: Nov. 25, 2010

(54) Method for Synthesizing and Using Pegylated Peptide-Photoactive Chromophore Conjugates and Micellular Formulations Thereof

(76) Inventor: Mark Savellano, White River Junction, VT (US)

Correspondence Address: LICATA & TYRRELL P.C., 66 E. MAIN STREET, MARLTON, NJ, 08053, US

(21) Appl. No.: 12/865,576

(22) PCT Filed: Jan. 30, 2009
(86) PCT No.: PCT/US09/32514

§ 371 Date: Aug. 2, 2010

Related U.S. Application Data
(60) Provisional application No. 61/025,020, filed on Jan. 31, 2008.

(51) Int. Cl. A61K 49/00; C07C 229/00; C07K 5/10; C07K 5/08; C07K 7/06; A61K 38/00; A61P 35/00

(52) U.S. Cl. 424/9.1; 530/331; 560/171; 530/330; 530/329; 530/328; 514/19.3

(57) ABSTRACT

The invention relates to a PEGylated peptide-chromophore conjugate, which forms irregular micelles, for use in photodiagnostic and phototherapeutic applications. Methods for synthesizing and using the conjugates of the invention are also provided.

### INTRODUCTION

This application claims benefit of priority from U.S. Provisional Patent Application Ser. No. 61/025,020, filed Jan. 31, 2008, the content of which is incorporated herein by reference in its entirety.

### BACKGROUND OF THE INVENTION

In the drug development process, many new drugs and diagnostic imaging agents that initially show great promise in in vitro studies often fail to work in vivo due to their limited bioavailability and/or poor pharmacodynamics in the body. This is especially true of many drugs/agents discovered in cancer research. In particular, many of the fluorescent chromophores (fluorophores) and photoactive chromophores (photosensitizers) used in photodiagnostic imaging and photodynamic therapy of malignancies and other pathologic lesions tend to be hydrophobic and/or lipophilic molecules, and typically exhibit a strong propensity to aggregate in aqueous solutions. Therefore, to administer such chromophores in vivo, the conventional approach has been to formulate them in solubilizers such as surfactants or liposomes or to derivatize them with peripheral sulfonic acid groups. While such formulations have shown a degree of success in the clinic, they are far from optimal and can have deleterious side effects. Specifically, solubilizing surfactants can be hemolytic and can cause allergic reactions. Moreover, surfactant micelles and liposomal vesicles are relatively large and are subject to instability due to interactions with serum proteins and disruption via simple dilution effects, so they tend to be rapidly cleared by the reticulo-endothelial and hepatobiliary systems. On the other hand, sulfonated chromophores are generally highly water soluble and do not suffer the aforementioned drawbacks of surfactant or liposomal formulations, but they can be rapidly cleared through the kidneys owing to their small molecular size. In addition, sulfonated chromophores can be extremely expensive because their production is often complicated and costly.

Photodiagnosis and phototherapy are well-developed fields of research, and there are numerous photodiagnostic/therapeutic applications that have already entered the clinic or are currently in clinical trials. Descriptions of chromophore-peptide/protein conjugate compositions, PEGylated chromophore compositions, and other types of water-solubilized chromophore compositions are described in the art (U.S. Pat. No. 5,238,940; U.S. Pat. No. 5,494,793; U.S. Pat. No. 5,543,514; U.S. Pat. No. 5,622,685; U.S. Pat. No. 6,036,941; U.S. Pat. No. 7,025,949; U.S. Patent Application No. 20020197262; WO 2003/079966; U.S. Pat. No. 6,554,853; U.S. Pat. No. 6,740,637; U.S. Pat. No. 6,949,581; U.S. Pat. No. 7,018,395; U.S. Pat. No. 6,083,485; U.S. Pat. No. RE38,994; Hamblin, et al. (2001) Cancer Res. 61:7155-7162; Jiang, et al. (2004) Proc. Natl. Acad. Sci. USA 101:17867-17872; Ferrera-Sinfreu, et al. (2005) J. Am. Chem. Soc. 127:9459-9468; Bettio, et al. (2005) Biomolecules 7:3534-3541; WO 2007/109364; Hans (2005) Ph.D. Thesis, Drexel University; Yokoyama, et al. (1991) Cancer Res. 51:3229-36; Yokoyama, et al. (1990) Cancer Res. 50:1693-700). Generally, these references teach compositions in which a PEG or other water-solubilizing group is directly or indirectly attached to the chromophore through covalent bonds, and the methods used to synthesize such compositions are typically highly specialized, complicated, and not easily adaptable to a variety of different chromophores.

### SUMMARY OF THE INVENTION

The present invention is a PEGylated peptide-chromophore conjugate composition of Formula II:

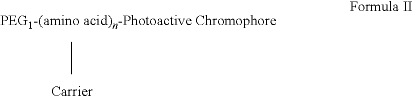

wherein PEG₁ is a linear PEG of 10 to 25 PEG units, n is 1 to 10, and the carrier is branched PEG. In certain embodiments, n of Formula I is 2 to 10, and at least one amino acid is conjugated to a branched PEG and one amino acid is conjugated to an active targeting carrier. Micellular formulations containing such conjugates are also embraced by the present invention as is a method for producing the conjugate of Formula II.

In this regard, the present invention is also a micellular formulation composed of at least one molecule of Formula I:

PEG₁-(amino acid)ₙ-Photoactive Chromophore Formula I

noncovalently associated with one molecule of Formula II:

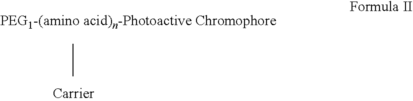

wherein PEG₁ is a linear PEG of 10 to 25 PEG units, and n is 1 to 10. While some embodiments embrace a micellular formulation wherein the carrier is a passive targeting carrier or an active targeting carrier, other embodiments embrace at least one amino acid conjugated to a passive targeting carrier and one amino acid conjugated to an active targeting carrier, wherein n is 2 to 10.

A method for producing the micellular formulations, consisting of a mixture of the components of Formula I and Formula II, is provided. Methods for diagnosing and treating lesions using the micellular formulations of the invention are also provided.

### BRIEF DESCRIPTION OF THE DRAWINGS

FIG. 1 depicts micellular PEGylated peptide-chromophore conjugate compositions. Chromophore, amino acids, PEG, and antibody molecules are indicated as are the covalent linkages between the antibody and conjugate. FIG. 1A depicts partially double-branched-PEGylated peptide conjugate for passive targeting. FIG. 1B shows an acetylated conjugate linked to a macromolecular carrier such as a monoclonal antibody for active targeting. Both covalently attached and noncovalently associated (via strong micellular amphiphilic interactions) conjugate components are shown. FIG. 1C depicts the production of an amphiphilic, irregular mixed micellular structure composed of a mixture of PEGylated peptide-chromophore conjugates. 1. A short linearly PEGylated peptide-chromophore conjugate is generated from basic building block components using solid phase synthetic chemistry. 2. The conjugate is further PEGylated via solution phase PEGylation to obtain a thoroughly dispersed formulation. 3. The PEGylated peptide-chromophore conjugate is further covalently conjugated to a macromolecular carrier such as an antibody to facilitate targeting. 4. PEGylated peptide-chromophore conjugates form self-assembling metastable micellular structures via noncovalent interactions.

FIG. 2 shows region of interest (ROI) analyses of fluorescence imaging data sets of A-431 tumor-bearing mice administered a composition of the invention. FIG. 2A shows data from a mouse injected with 60 nmoles of surfactant-solubilized free pyropheophorbide-a (PPa) in an excipient mixture of 2% ethanol/1% TWEEN 80/PBS. FIG. 2B shows data from a mouse injected with 60 nmoles PPa content of a partially PEG2(20 kDa)ylated (˜63 molar %) short PEGylated PPa peptide (sPPp) conjugate in PBS. FIG. 2C shows data from a mouse injected with 10 nmoles PPa content of sPPp-ERBITUX® (˜11.5 sPPp/Monoclonal antibody, ˜42 molar % sPPp noncovalently bound) in PBS. ROI measurements were done using ImageJ software (Rasband 1997-2007). For FIGS. 2A and 2B, image exposure times were 0.65 seconds, whereas image exposure times in FIG. 2C were 4 seconds in order to compensate for the smaller injected dosage of PPa content.

FIG. 3 shows tumor growth delay studies of various phototherapeutic treatments. Mice were implanted with A-431 tumor xenografts and were administered phototherapy when their tumor sizes reached approximately 150 mm³. Tumors were then regularly measured to determine the time it took for the tumors to reach four times their initial treatment size. To allow for easier comparisons, tumor growth delay data sets have been aligned so that the 0-day time point corresponds to a tumor volume of 150 mm³. Diamond, control mice (no PPa or sPPp conjugate; no light), lumped data set, n=6. Squares, mice treated with 60 nmoles surfactant-solubilized free PPa in 2% ethanol/1% TWEEN 80/PBS for a ˜16 hour incubation and treated with 100 J/(cm*cm) light, n=4. Triangles, mice treated with 60 nmoles partially PEG2(20 kDa)ylated (˜63 molar %) sPPp conjugate in PBS for a ˜16 hour incubation and treated with 500 J/(cm*cm) light, n=3.

FIG. 4 shows graphs of tumor growth delay studies of various single-treatment photodynamic therapy (PDT) regimens using a conventional surfactant-solubilized pyropheophorbide-a (PPa) formulation in 2% ethanol/1% Tween 80/5% dextrose (FIG. 4A), an acetylated-sPPp (Ac-sPPp) formulation (FIG. 4B), and an anti-epidermal growth factor receptor (EGFR)-targeted sPPp immunoconjugate, ERBITUX-sPPp (FIG. 4C). All formulations were administered at a dosage equivalent to 60 nmole PPa content per 20 gram body weight in a 200 μl bolus injection via the tail vein (or via retro-orbital injection when tail vein injections were not feasible). A 670 nm diode laser light dose of 100 J/cm², delivered at ˜50 mW/cm², was given at ˜16 hours post-injection over an area covering the entire tumor surface as well as a 2 to 3 mm border extending beyond the edge of the tumor. The tumor model was the EGFR-overexpressing A-431 tumor xenograft grown in athymic NCr nu/nu mice (5-week old mice from the National Cancer Institute at Frederick, Md.). Data series were plotted so that the time at which each tumor reached ˜150 mm³ corresponds to 0 days; i.e., the approximate time at which the PDT light dose was administered.

FIG. 5 shows graphs of tumor growth delay studies of repeat-treatment PDT regimens using two PDT treatments with an acetylated-sPPp (Ac-sPPp) formulation (FIG. 5A) and two PDT treatments with an anti-EGFR-targeted sPPp immunoconjugate, ERBITUX-sPPp (FIG. 5B). The tumor model, formulation dosages, and light dose administrations were the same as described in FIG. 4. Arrows in the graphs indicate the time points at which the second PDT treatment was given for each mouse.

### DETAILED DESCRIPTION OF THE INVENTION

To overcome problems of solubility, a novel approach for formulating chromophores has been developed that optimizes bioavailability and pharmacodynamics for in vivo photodiagnostic and phototherapeutic applications without using surfactants or employing costly and complicated derivatizations of the chromophore (e.g., attachment of peripheral sulfonic acid groups to the chromophore macrocycle). Specifically, the present invention relates to the solubilization and dispersion of a chromophore via peptide conjugation and stepwise polyethylene glycolation (PEGylation; i.e., conjugation with polyethylene glycol (PEG)). Advantageously, the PEGylated peptide-photoactive chromophore conjugate of the present invention can form a metastable micellular structure which can be delivered through the bloodstream without significantly coming apart due to interaction with serum proteins. Therefore, the present composition can reach the tumor environment in greater yield with less non-specific deposition in normal tissue.

Generally, the present invention relates to the conjugation of photoactive chromophore with a short peptide (e.g., a peptide of 1 to 10 amino acid residues) and a dispersive solubilizing polymer. More specifically, the present invention provides conjugation of a photoactive chromophore to a peptide and PEG thereby providing the composition of Formula I:

PEG₁-(amino acid)ₙ-Photoactive Chromophore Formula I

wherein PEG₁ is 10-25 linear polyethylene glycol units and n is 1 to 10.

As is conventional in the art, an “amino acid” refers to the basic chemical structural unit of a protein or polypeptide. In accordance with the present invention, an amino acid includes a naturally occurring amino acid as well as derivatives thereof. Naturally occurring amino acids include alanine (Ala or A), arginine (Arg or R), asparagine (Asn or N), aspartic acid (Asp or D), Cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V). An amino acid derivative denotes an amino acid residue which is not naturally incorporated into a polypeptide chain during protein biosynthesis, i.e., during translation. In this regard, an amino acid derivative is not proteinogenic. Amino acid derivatives include amino acid residues modified by post-translation modification (e.g., acetylation, amidation, formylation, hydroxylation, methylation, phosphorylation, or sulfatation) as well as D-amino acid residues and other non-proteinogenic amino acid residues such as 2-Aminoadipic acid, 3-Aminoadipic acid, beta-Alanine, beta-Aminoproprionic acid, 2-Aminobutyric acid, 4-Aminobutyric acid, Piperidinic acid, 6-Aminocaproic acid, 2-Aminoheptanoic acid, 2-Aminoisobutyric acid, 3-Aminoisobutyric acid, 2-Aminopimelic acid, t-butylalanine, Citrulline, Cyclohexylalanine, 2,4-Diaminobutyric acid, Desmosine, 2,2′-Diaminopimelic acid, 2,3-Diaminoproprionic acid, N-Ethylglycine, N-Ethylasparagine, Homoarginine, Homocysteine, Homoserine, Hydroxylysine, Allo-Hydroxylysine, 3-Hydroxyproline, 4-Hydroxyproline, Isodesmosine, allo-Isoleucine, Methionine sulfoxide, N-Methylglycine, sarcosine, N-Methylisoleucine, 6-N-Methyllysine, N-Methylvaline, 2-Naphthylalanine, Norvaline, Norleucine, Ornithine, 4-Chlorophenylalanine, 2-Fluorophenylalanine, 3-Fluorophenylalanine, 4-Fluorophenylalanine, Phenylglycine, Beta-2-thienylalanine, as well as peptide nucleic acids. In certain embodiments, one or more amino acid residues are selected for containing a functional group (e.g., a side chain amino or sulfhydryl group) to facilitate conjugation with PEG and/or other carrier. In certain embodiments, one or more amino acid residues of the instant composition are lysine. In particular embodiments, the instant composition is composed of Asp and Lys.

The number of amino acid residues employed in the instant composition can vary. However, in particular embodiments, a short peptide is desirable, e.g., a peptide of 1 to 10 amino acid residues. In this regard, the number of amino acids employed can be 1, 2, 3, 4, 5, 6, 7, 8, 9 or 10 amino acids.

In some embodiments, the peptide amino acid sequence is selected to resemble or mimic a ligand that binds preferentially to an overexpressed or overactive oncogenic receptor, or is selected to resemble or mimic an enzyme substrate that can be cleaved by a tumor-associated enzyme (e.g., an MMP-1 protease cleavage sequence), thereby allowing for targeted drug release in the vicinity of a tumor. In photodiagnostic/therapeutic applications, a potential added benefit of such an enzyme substrate is that it can be rendered dequenchable if a quencher dye is attached in an appropriate manner to the peptide-chromophore conjugate (Weissleder, et al. (1999) Nat. Biotechnol. 17:375-8).

As used herein, a photoactive chromophore, also commonly referred to as a photosensitizer, is a compound activated by continuous wave or pulsed coherent or incoherent electromagnetic radiation having a wavelength in the range from about 400 nm to about 800 nm. The parameters of the coherent or incoherent electromagnetic radiation preferably are selected so that the radiation is capable of penetrating the tissue to a certain depth, activating the photosensitizer, and producing phototoxic damage in the targeted diseased tissue.

Photoactive chromophores useful in the practice of the invention include, for example, chlorins, cyanines, purpurins and porphyrins, for example, benzoporphyrin derivative monoacid (BPD-MA) (available from QLT, Inc., Vancouver, Canada). Other useful photoactive chromophores include, for example, bacteriochlorins and bacteriopurpurins, such as those described in U.S. Pat. No. 6,376,483, for example 5,10-octaethylbacteriopurpurin, and 5,15-octaethylbacteriopurpurin, or nickel 5,10-bis-acrylate etioporphyrin I. Other useful photoactive chromophores include xanthenes, for example, rose bengal, or other photosensitizers that may be isolated or derived from natural sources, or synthesized de novo, for example, hypericin (available from Sigma Chemical Co., St. Louis, Mo.). See also photoactive chromophores disclosed in WO 2003/07996 and U.S. Pat. Nos. 6,036,941; 6,740,637; and RE38,994. It is understood that this list of photoactive chromophores including those in Table 1 is exemplary, and that other photoactive chromophores having the appropriate spectral characteristics can also be useful in the practice of the invention.

TABLE 1

| | Approximate wavelength |
|---|---|
| | of selected absorption |
| Photosensitizer | peaks (nm) |
| chlorin e6 | 410, 658 |
| mono-N-aspartyl chlorin e6 | 664 |
| tin etiopurpurin | 660 |
| lutetium texaphyrin | 470, 730 |
| 5,10-octaethylbacteriopurpurin | 563, 598 |
| 5,15-octaethylbacteriopurpurin | 558, 592 |
| nickel 5,10-bis-acrylate | 580 |
| etioporphyrin I | |
| protoporphyrin IX | 506, 546, 578, 635 |
| benzoporphyrin derivative monoacid | 400, 585, 687 |
| hypericin | 550, 595 |
| rose bengal | 548 |
| hematoporphyrin derivative | 505, 537, 565 |
| Pd(II)-octabutoxyphthalocyanine | 732, 838 |
| Si(IV)-naphthocyanine | 773 |
| Pyropheophorbide-a | 660 |

The invention also encompasses pro-photosensitizers, which when administered to a mammal are capable of being metabolized or otherwise converted to produce a photoactive chromophore, or are capable of stimulating the synthesis of an endogenous photoactive chromophore. It is contemplated that the pro-photosensitizer may be converted into a photosensitizer of interest or may stimulate the synthesis of an endogenous photosensitizer at the site to be treated. Alternatively, the pro-photosensitizer can be converted into a photosensitizer or stimulate the synthesis of an endogenous photosensitizer at a region remote from the target region, after which the photosensitizer is transported to the target skin region, for example, via the vasculature. When a pro-photosensitizer is used, the pro-photosensitizer is allowed to accumulate, metabolize, covert, or otherwise stimulate the synthesis of a photoactive chromophore.

It is contemplated that pro-photosensitizers useful in the practice of the invention include, for example, precursors of PpIX, for example, ALA (available from Sigma Chemical Co., St. Louis, Mo.), ALA derivatives, such as, ALA esters (e.g., ALA-methyl ester, ALA-n-pentyl ester, ALA-n-octyl ester, R,S-ALA-2-(hydroxymethyl)tetrahydropyranyl ester, N-acetyl-ALA, and N-acetyl-ALA-ethyl ester). See, e.g., U.S. Pat. No. 6,034,267.

Conjugation of photoactive chromophores to peptides, proteins, and various synthetic polymers is routinely carried out by the skilled artisan and any suitable method can be employed to conjugate a photoactive chromophore to the carboxy or amino terminal amino acid of the instant conjugate. See, e.g., Hamblin, et al. (2001) Cancer Res. 61:7155-7162; Jiang, et al. (2004) Proc. Natl. Acad. Sci. USA 101:17867-17872; and Bettio, et al. (2005) Biomacromolecules 7:3534-3541.

Polyethylene glycol (PEG), in its most common form, is a linear polymer having hydroxyl groups at each terminus: HO—CH₂—CH₂O(CH₂CH₂O)ₙCH₂CH₂—OH, wherein CH₂CH₂O represents the repeating monomer unit of PEG. In accordance with the present invention, a short linear PEG (PEG₁) is attached to the amino acid carboxy or amino terminus. While the PEG₁ compound can itself be quite varied in composition, PEG₁ contains from 10 to 25 units of PEG monomers, i.e., (—CH₂CH₂O—)ₙ, wherein n is 10 to 25. In particular embodiments, PEG₁ is linked or attached to the carboxy or amino terminal amino acid via solid phase synthesis, e.g., by employing PEG building blocks such as O—(N-Fmoc-2-aminoethyl)-O′-(2-carboxyethyl)-undecaethylene glycol available from commercial sources such as EMD Biosciences (La Jolla, Calif.). Solid phase synthesis of the PEG-peptide-chromophore conjugate advantageously allows direct attachment of the amino acid to the PEG.

The enhanced solubility provided by PEG₁ not only improves the yield of the conjugate following standard solid phase synthesis and reversed-phase purification procedures but also makes subsequent conjugate manipulations much easier, since stringent organic solvents are no longer required to prevent the inherently hydrophobic/lipophilic chromophore from forming large aggregates that tend to precipitate out of solution. However, for some targeting applications, a short linearly PEGylated peptide-chromophore conjugate requires one or more additional carriers to obtain an even more thoroughly dispersed formulation.

Accordingly, a PEGylated peptide-chromophore conjugate of the invention can further include a targeting carrier. A targeting carrier in the context of the present invention is a molecule which facilitates delivery of the PEGylated peptide-chromophore conjugate to the site of action. A PEGylated peptide-chromophore conjugate with a targeting carrier is represented by Formula II:

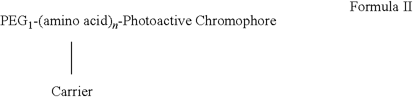

wherein PEG₁ is 10-25 linear polyethylene glycol units, n is to 10, and “|” represents a covalent bond to the targeting carrier. Depending on the application and/or desired effect, a carrier of the invention can be a passive targeting carrier or an active targeting carrier.

In certain embodiments of the present invention, the targeting carrier is a passive targeting carrier. In particular embodiments, the passive targeting carrier is a branched PEG. The branched PEG can be represented as R(-PEG-OH)ₘ in which R represents a central core moiety such as pentaerythritol or glycerol, and m represents the number of branching arms. The number of branching arms (m) can range from one, two, three, four, five, six, seven, eight, nine, or up to 10 or more. Moreover, the molecular weight of the branched PEG can range from 10 kDa to 2,000 kDa. In particular, the analysis disclosed herein indicates that a double branched PEG of at least 20 kDa decreased loss of conjugates through the kidneys thereby increasing circulation time. Accordingly, some embodiments of the present invention provide a double branched PEG with a size of at least 20 kDa.

Also within the context of a branched PEG is that described in PCT patent application WO 96/21469, which has a single terminus that is subject to chemical modification. This type of PEG can be represented as (CH₃O-PEG-)ₚR—X, whereby p equals 2 or 3, R represents a central core such as lysine or glycerol, and X represents a functional group such as carboxyl that is subject to chemical activation. Yet another branched form, the “pendant PEG”, has reactive groups, such as carboxyl, along the PEG backbone rather than at the end of PEG chains.

In addition to these forms of PEG, PEG as a passive targeting carrier can also be prepared with weak or degradable linkages in the backbone. For example, PEG can be prepared with ester linkages in the polymer backbone that are subject to hydrolysis. This hydrolysis results in cleavage of the polymer into fragments of lower molecular weight.

The passive targeting carrier of the present composition is desirably covalently linked or attached to one or more amino acid residue side chains (e.g., an amino or sulfhydryl group) of the peptide-photoactive chromophore conjugate in solution phase. Since a multitude of solution phase compatible PEGylation reagents are commercially available (e.g., from suppliers such as Nektar Therapeutics, Huntsville, Ala. and NOF Corporation, Tokyo, Japan), wherein said PEGs have a variety of activated groups and PEGs of different sizes and configurations, variable types of PEG moieties can be employed as passive targeting carriers. Examples of such PEGs include PEG activated esters, PEG aldehyde, PEG epoxide or PEG tresylate.

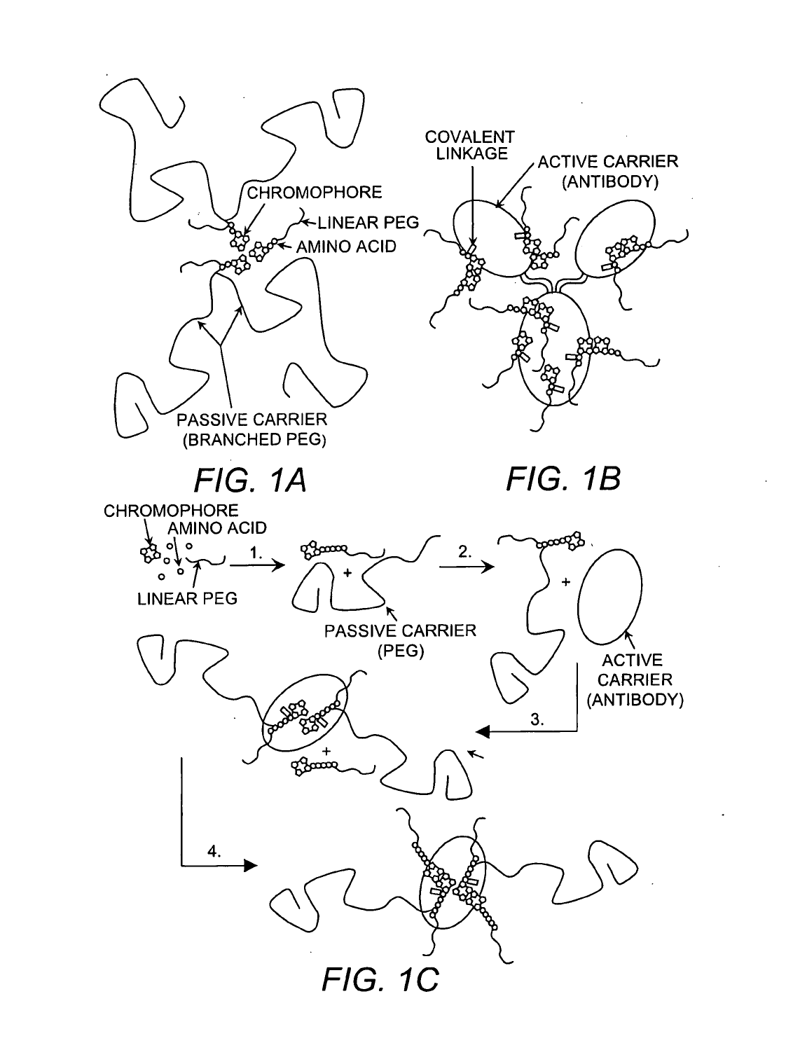

PEGylation of a peptide-active chromophore conjugate with a short linear PEG (PEG₁) at a PEG₁:(amino acid)ₙ-active chromophore ratio of 1 and a large branched PEG as a passive targeting carrier at a carrier:PEG₁-(amino acid)ₙ-Photoactive Chromophore molar ratio of <1 results in a highly amphiphilic, irregular mixed micellular structure (see FIG. 1A), which exhibits favorable pharmacokinetics/distribution. Advantageously, PEGylation in the manner set forth in the present invention improves the in vivo activity of photoactive chromophore. Indeed, PEGylated drugs exhibit decreased immunogenicity and antigenicity, slower rates of undesirable enzymatic degradation reactions, dramatically reduced renal/cellular/and reticulo-endothelial system (RES) clearances, improved solubility, and generally longer blood circulation half-lives (Putnam (1995) Adv. Polymer Sci. 122:55-123; Parveen & Sahoo (2006) Clin. Pharmacokinet. 45:965-88).

For some applications, the above described PEGylated peptide-photoactive chromophore conjugates provide the desired targeting properties by taking advantage of the enhanced permeability and retention (EPR) effect (Matsumura & Maeda (1986) Cancer Res. 46:6387-92). However, in accordance with some embodiments, the PEGylated peptide-chromophore conjugate is modified by covalent conjugation to an active targeting carrier or moiety (e.g., a small molecule ligand such as folic acid or tumor-targeting antibody) to promote active targeting to disease-associated molecular targets (e.g., antibody targeting of overexpressed growth factor receptors on tumor cells). This can be accomplished via activation of a side chain residue on the PEGylated peptide-chromophore conjugate (e.g., conversion of an amino acid side chain carboxyl group to an activated ester or reduction of a disulfide bond to a labile sulfhydryl) followed by conjugation of the activated residue to complementary groups on the active targeting carrier or moiety molecule (see FIGS. 1B and 1C).

As used herein, an active targeting carrier is any molecule that can be covalently conjugated to a functional group of the instant conjugate to facilitate, enhance, or increase the transport of the conjugate to or into a target cell, tissue, or structure (e.g., a cancer cell, an immune cell, a pathogen, the brain, etc.) by an active mechanism. Active targeting carriers include polypeptides, peptides, antibodies, antibody fragments, oligonucleotide-based aptamers with recognition pockets, and small molecules that bind to disease-associated molecular targets such as specific cell surface receptors or polypeptides on the outer surface of the cell wherein the cell surface receptors or polypeptides are specific to that cell type. For example, a variety of protein transduction domains, including the HIV-1 Tat transcription factor, Drosophila Antennapedia transcription factor, as well as the herpes simplex virus VP22 protein have been shown to facilitate transport of proteins into the cell (Wadia and Dowdy (2002) Curr. Opin. Biotechnol. 13:52-56). Further, an arginine-rich peptide (Futaki (2002) Int. J. Pharm. 245:1-7), a polylysine peptide containing Tat PTD (Hashida, et al. (2004) Br. J. Cancer 90(6):1252-8), PTD-4 (Ho, et al. (2001) Cancer Res. 61:474-477), transportin (Schwartz and Zhang (2000) Curr. Opin. Mol. Ther. 2:2), Pep-1 (Deshayes, et al. (2004) Biochemistry 43(6):1449-57) or an HSP70 protein or fragment thereof (WO 00/31113) is suitable for targeting a conjugate of the present invention. Not to be bound by theory, it is believed that such transport domains are highly basic and appear to interact strongly with the plasma membrane and subsequently enter cells via endocytosis (Wadia, et al. (2004) Nat. Med. 10:310-315).

Moreover, peptide hormones such as bombesin, stomatostatin and luteinizing hormone-releasing hormone (LHRH) or analogs thereof can be used as active targeting carriers. Cell-surface receptors for peptide hormones have been shown to be overexpressed in tumor cells (Schally (1994) Anti-Cancer Drugs 5:115-130; Lamharzi, et al. (1998) Int. J. Oncol. 12:671-675) and the ligands to these receptors are known tumor cell targeting agents (Grundker, et al. (2002) Am. J. Obstet. Gynecol. 187(3):528-37; WO 97/19954). Carbohydrates such as dextran having branched galactose units (Ohya, et al. (2001) Biomacromolecules 2(3):927-33), lectins (Woodley (2000) J. Drug Target. 7(5):325-33), and neoglycoconjugates such as Fucalpha1-2Gal (Galanina, et al. (1998) Int. J. Cancer 76(1):136-40) may also be used as active targeting carriers to treat, for example, colon cancer. It is further contemplated that an antibody or antibody fragment which binds to a protein or receptor, which is specific to or overexpressed on a tumor cell, can be used as an active targeting carrier. Preferably, the antibody fragment retains at least a significant portion of the full-length antibody's specific binding ability. Examples of antibody fragments include, but are not limited to, Fab, Fab′, F(ab′)₂, scFv, Fv, dsFv diabody, or Fd fragments. Exemplary antibody carriers include an anti-HER-2 antibody (Yamanaka, et al. (1993) Hum. Pathol. 24:1127-34; Stancovski, et al. (1994) Cancer Treat Res. 71:161-191) for targeting breast cancer cells and bispecific monoclonal antibodies composed of an anti-histamine-succinyl-glycine Fab′ covalently coupled with an Fab′ of either an anticarcinoembryonic antigen or an anticolon-specific antigen-p antibody (Sharkey, et al. (2003) Cancer Res. 63(2):354-63).

Transferrin is another suitable active targeting carrier which has been extensively investigated as a ligand for targeting of antineoplastic agents (Qian, et al. (2002) Pharmacol. Rev. 54:561-587; Widera, et al. (2003) Adv. Drug. Deliv. Rev. 55:1439-1466). Moreover, transferrin has been used to deliver therapeutic agents across the blood-brain barrier, which is otherwise impermeable to most therapeutic agents (Pardridge (2002) Adv. Exp. Med. Biol. 513:397-430; Bickel, et al. (2001) Adv. Drug Deliv. Rev. 46:247-279).

Standard methods employing homobifunctional or heterobifunctional crosslinking reagents such as carbodiimides, sulfo-NHS esters linkers, and the like can be used for conjugating or operably attaching the active carrier to a functional group of the conjugate of the present invention, as can aldehyde crosslinking reagents, such as glutaraldehyde.

It is contemplated that the PEGylated peptide-chromophore conjugates of the present invention can be modified with one type of carrier or a plurality of carriers. For example, two amino acid residues of the peptide of a PEGylated peptide-chromophore conjugate can be conjugated to two different carriers, e.g., two different active targeting carriers; two different passive targeting carriers; or one active and one passive targeting carrier. For example, in the event of the latter, n in the conjugate of Formula II:

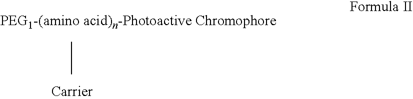

would be 2 to 10, wherein at least one amino acid is conjugated to a passive targeting carrier and one amino acid is conjugated to an active targeting carrier; PEG₁ is 10-25 linear polyethylene glycol units; and “|” represents a covalent bond to the carriers. Also within the scope of the present invention is a composition wherein a plurality (e.g., two or more) of PEGylated peptide-chromophore conjugate molecules is attached to a single carrier molecule (e.g., an antibody). See, for example, FIGS. 1B and 1C.

In an advantageous variation of the present invention, the targeting carrier is a macromolecule and the PEGylated peptide-chromophore conjugate possesses substantial amphiphilicity/amphipathicity. Under these circumstances, self-assembling metastable micellular conjugate structures can form from a mixture of PEGylated peptide-chromophore conjugates covalently conjugated to a carrier (i.e., Formula II) and free noncovalently associated PEGylated peptide-chromophore conjugates (i.e., Formula I). See FIGS. 1A-1C. Such self-assembling metastable micellular conjugate formulations are another salient feature of this invention. In effect, these formulations are possible because the amphiphilic PEGylated peptide-chromophore conjugate essentially acts as its own surfactant-like solubilizer. Distinct advantages of the metastable micellular conjugate formulations include: highly effective shielding of the hydrophobic/lipophilic chromophore within the compact core of a micelle structure and consequently, much improved solubility; larger chromophore payloads per carrier; slow but virtually guaranteed degradation of the metastable structures in vivo ensuring prolonged stability during delivery and excellent biocompatibility; and overall much improved bioavailability and pharmacodynamics.

PEGylated peptide-chromophore conjugates and micelles of the present invention find use in a variety of applications. In particular, the instant conjugates and micelles find application in photodiagnosis and photodynamic therapy of lesions such as cancer. In addition, photodynamic therapy has shown the potential to treat several other types of conditions including psoriasis, arthritis, atherosclerosis and purifying blood infected with viruses, including HIV. Furthermore, the treatment of age-related macular degeneration (AMD) and microbial infections are already in clinical use or are current areas of research (Pandey (2000) J. Porphyrins Phthalocyanines 4:368-373).

It is considered that the choice of the appropriate photoactive chromophore or pro-photosensitizer, dosage, and mode of administration will vary depending upon several factors including, for example, the lesion or condition to be treated, and the age, sex, weight, and size of the mammal to be treated, and may be varied or adjusted according to choice. The instant conjugate composition is administered to a subject having or at risk of a lesion or condition so as to permit an effective amount of photoactive chromophore to be present in the target region upon application of an appropriate light dose. A subject having a lesion or condition, in general, exhibits one or more signs associated with the lesion or condition. A subject at risk of a lesion or condition is intended to include a subject that has a familial history of the lesion or condition or due to other circumstances may be predisposed to develop the lesion or condition. For example, a patient at risk of developing a lesion such as cancer would include a subject that has a family history of cancer or has been exposed to a cancer-causing agent.

As used herein, the term “effective amount” means an amount of photoactive chromophore suitable for photodynamic therapy, i.e., the photoactive chromophore is present in an amount sufficient to produce a desired photodynamic reaction at the target site. For example, an effective amount is considered an amount that causes a measurable change in one or more signs or symptoms associated with the select lesion or condition when compared to otherwise same lesions or conditions wherein the conjugate is not present. For example, an effective amount of PEGylated peptide-photoactive chromophore conjugate or micelle in the treatment of cancer would cause a measurable decrease in hyperplasia or cell proliferation as compared to cells not exposed to the conjugate or micelle. Further, an effective amount as an antibiotic would result in an inhibition or decrease in the number of viable bacterial, fungal, or protozoan cells.

The PEGylated peptide-chromophore conjugate or micelle formulation can be administered in a single dose or multiple doses over a period of time to permit an effective amount of photoactive chromophore to accumulate in the target region. Fluorescence spectroscopy or other optical detection or imaging techniques can be used to determine whether and how much photoactive chromophore is present in the target region.

Desirably the photoactive chromophore mitigates, cures, treats or prevents the lesion or condition, e.g., cancer. It is particularly desirable that the photoactive chromophore be capable of exerting an effect locally (i.e., at or near the site of the disease or condition).

Conjugate compositions or micelle formulations of the present invention can be administered either alone, or in combination with a pharmaceutically or physiologically acceptable carrier, excipient or diluent. Generally, such carriers should be nontoxic to recipients at the dosages and concentrations employed. Ordinarily, the preparation of such compositions entails combining the conjugate composition or micelle formulations of the present invention with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. The type of carrier, excipient or diluent employed can be dependent upon the lesion or condition being treated and the route of administration. It is contemplated that the instant conjugate composition or micelle formulations can be administered via any conventional route including intralesional, or subcutaneous, intradermal, intramuscular, intraocular, or intra-articular injection, and the like. Further, the conjugate composition or micelle formulations of the invention can also be applied to the skin using any of the known methodology, for example in the form of creams, ointments, emulsions, or solutions.

It should be noted that the photoactive chromophore or pro-photosensitizer dosage should be adjusted with respect to the irradiation parameters, including, for example, wavelength, fluence, fluence rate, irradiance, duration of the light, and the time interval between administration of the photoactive chromophore or pro-photosensitizer and the irradiation, and the cooling parameters, if surface cooling is necessary or desired. All of these parameters should be adjusted to produce a photodynamic reaction resulting from activation of the photoactive chromophore in the target region that is effective with minimal side effects. Such considerations and adjustments are routinely practiced in the art.

Suitable light sources useful for activating the chromophore of the invention include incoherent light sources, optionally with one or more light filters, and coherent light sources. Suitable incoherent light sources include, for example, flash lamps and filtered flash lamps. Suitable coherent light sources include, for example, pulsed lasers, e.g., pulsed diode lasers such as gallium arsenide diode lasers and flashlamp pumped pulsed dye lasers. Other suitable pulsed lasers include pulsed solid state lasers, for example flashlamp pumped alexandrite lasers and neodymium:YAG lasers. Other suitable coherent light sources include cw lasers that are scanned, beam-expanded, or diffused over and/or into the treatment area/volume, for example, cw dye lasers, frequency doubled neodymium:YAG lasers, or cw diode lasers.

A novel and versatile method for producing a surfactant-free chromophore formulation that exhibits optimal bioavailability and favorable pharmacodynamics for in vivo photodiagnostic/therapeutic applications has been developed (see FIG. 1C). The fundamental component of the formulation is a chromophore conjugated to a polyethylene glycolated (PEGylated) oligopeptide. The PEGylated peptide-chromophore conjugate can be further functionalized by conjugation to a passive targeting carrier (e.g., a macromolecular branched PEG) and, or in the alternate, an active targeting carrier (e.g., an antibody). In addition, if the chromophore is significantly hydrophobic/lipophilic, the PEGylated peptide-chromophore conjugate may be highly amphiphilic and assemble into micellular compositions under appropriate conditions. Such micellular compositions have distinct advantages due to their solubility, compactness, high dispersity, and metastable nature. Accordingly, the instant formulation can be used as an alternative method for efficient in vivo delivery of small molecule drugs/agents that exhibit poor bioavailability, which is especially the case for drugs and agents that are highly hydrophobic/lipophilic. The PEGylated peptide delivery vehicles and their micellular compositions described herein can be used to salvage many small molecule drugs/agents that have Shown great efficacy in vitro but that have otherwise failed in in vivo studies and/or have required potentially harmful surfactants for in vivo use. In particular, the invention is well suited for in vivo delivery of fluorophore and photosensitizer chromophores for use in photodiagnosis and photodynamic therapy.

The invention is described in greater detail by the following non-limiting examples.

### Example 1

### Synthesis of PEGylated Peptide-Chromophore Conjugate

Conjugate formulations composed of the fluorescent photosensitizer chromophore, pyropheophorbide-a (PPa), were prepared. However, as the skilled artisan can appreciate, other fluorophore or photosensitizer chromophores can be formulated in a similar manner. A short linear PEG strand constructed from a linkage of two undecaethyleneglycol building blocks was employed for the first PEGylation step in the assembly of the PPa-peptide conjugates. With respect to the amino acid sequence of the peptide component, a two amino acid peptide, N-epsilon(Asp)-Lys was employed. However, it is contemplated that longer peptides (e.g., up to 10 amino acid residues) incorporating protease cleavage sites or receptor binding sequences can also be used.

The chemical structure of the resulting short linearly PEGylated peptide-chromophore conjugate was:

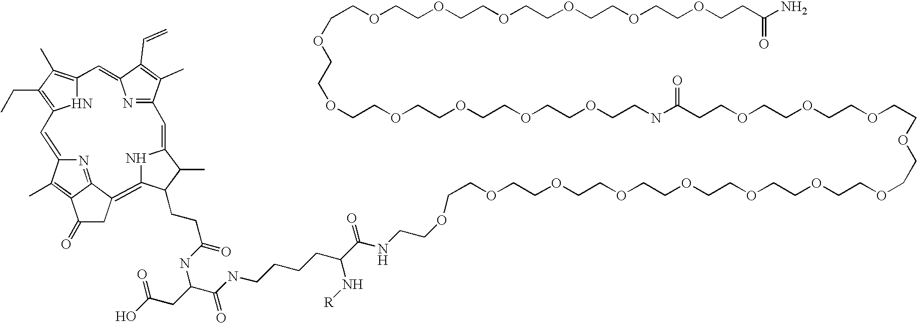

For the studies presented herein, R was —H, a PEG side chain, or an acetyl (—COCH₃). For R═H, the conjugate was designated ‘sPPp’ (short PEGylated PPa-peptide).

The N-epsilon(Asp)-Lys peptide was of particular use because it is one of the simplest and most versatile constructs that can be built. Positioning the hydrophobic PPa chromophore at the amino terminus Asp residue with the short linear PEG strand attached at the peptide carboxy terminus gives a highly amphiphilic/amphipathic species. When a more thoroughly dispersed sPPp conjugate formulation was required, the alpha-amino group of the Lys residue was modified with a second PEG group using simple active ester conjugation chemistry. Furthermore, the gamma-carboxy group of the Asp residue could be converted to an active ester to allow covalent attachment of the sPPp conjugate to a targeting moiety or carrier.

The sPPp conjugate was highly soluble and readily dissolved in aqueous solutions. In comparison, aqueous solutions of free PPa required potentially harmful solubilizers to prevent the formation of large insoluble aggregates (e.g., an excipient mixture of 2% ethanol and 1% TWEEN 80 was used in clinical trials with HPPH (2-[1-hexyloxyethyl]-2-devinyl pyropheophorbide-a), a closely related derivative of PPa (Bellnier, et al. (2003) Cancer Res. 63:1806-13). However, in aqueous solutions, the fluorescence of the sPPp conjugate was weak due to static concentration quenching effects, indicating that the sPPp conjugate tended to form large micelles. Moreover, dynamic light scattering measurements suggested that the sPPp micelles were very heterogeneous in size. Based on these observations, it was concluded that the sPPp conjugate, by itself without further modification, would most likely be suboptimal for in vivo applications. Therefore, two different approaches for modifying the sPPp conjugate were investigated to improve its in vivo targetability.

For the first approach, it was demonstrated that attaching a second PEG group to the conjugate would provide a more thoroughly dispersed formulation for passive targeting via the EPR effect. Using active ester chemistry, it was found that the attachment of double-branched PEGs to the Lys alpha-amino group of the sPPp conjugate almost fully dequenched the sPPp conjugate, whereas the attachment of single-branched PEGS only moderately dequenched the conjugate. Moreover, it was observed that sPPp conjugate formulations that had only been partially PEGylated with a double-branched PEG (e.g., ˜63 molar percent double-branched PEGylated sPPp mixed with ˜37 molar percent sPPp, as determined by SDS-PAGE analysis) were still highly dequenched. These results indicate that partially double-branched-PEGylated sPPp formulations form highly dispersed irregular micellular structures, which are held together via strong noncovalent amphiphilic interactions. The depiction of a micellular structure with partially double-branched-PEGylated sPPp conjugates is shown in FIG. 1A. With respect to the size of the double-branched PEG, it was observed in pilot imaging studies that a 10 kDa double-branched PEGylated conjugate could be cleared rather extensively through the kidneys. When the size was increased to 20 kDa, significantly less conjugate was lost through the kidneys, and the conjugate had a noticeably longer circulation time. Consequently, a 20 kDa double-branched PEG (abbreviated PEG2(20 kDa)) was employed in the in vivo experimental results disclosed herein.

For the second approach, it was investigated whether attaching the sPPp conjugate to an antibody would enable active targeting. Specifically, the Lys alpha-amino group of the sPPp conjugate was acetylated and the Asp gamma-carboxy group was converted to an active ester. The resulting acetylated sPPp active ester was conjugated to the lysine residues of a commercial therapeutic anti-epidermal growth factor receptor (EGFR) monoclonal antibody, ERBITUX® (Garber (2000) J. Natl. Cancer Inst. 92:1462-4). It was observed that the ERBITUX® monoclonal antibody could be labeled with as many as ˜11.5 sPPp per monoclonal antibody. However, up to ˜42 molar percent of the sPPp was not covalently attached and was instead associated through very strong noncovalent amphiphilic interactions (as determined by SDS-PAGE analysis), which could not be disrupted even after rigorous gel filtration. It was also observed that the sPPp-ERBITUX® conjugate formulations were strongly quenched. Taken as a whole, these observations indicate that the sPPp-ERBITUX® conjugate formulations were composed of sPPp covalently attached to the monoclonal antibody with additional sPPp noncovalently associated via strong amphiphilic interactions around the conjugate, forming an irregular micellular configuration. FIG. 1B depicts a micellular structure of such a sPPp-monoclonal antibody conjugate formulation.

### Example 2

### In Vivo Delivery Characteristics of PEGylated Peptide-Chromophore Conjugate

To assess the in vivo delivery characteristics of sPPp conjugate formulations, time-coursed fluorescence imaging studies were performed in a tumor xenograft model grown in athymic nude mice. Tumor xenografts were grown subcutaneously in the left upper chest region using the A-431 human epidermoid carcinoma cell line (American Type Culture Collection, Manassas, Va.), which is ideal for EGFR-targeting studies given that these cells overexpress ˜1×10⁶-2.6×10⁶ EGFR/cell (Haigler, et al. (1978) Proc. Natl. Acad. Sci. USA 75:3317-21; Mendelsohn (1997) Clin. Cancer Res. 3:2703-7). When tumor sizes reached greater than ˜100 mm³, mice were imaged pre-injection, and then free PPa or sPPp conjugate formulations that had been sterile filtered through a 0.2 micron membrane were injected via tail vein or retro-orbitally at dosages ranging from ˜10 to ˜80 nmoles PPa content per mouse (mice weighed ˜20 g) in a single bolus volume of approximately 200 microliters. A series of post-injection images were then taken over a period of time until mouse fluorescence levels approached pre-injection baseline levels. Images were acquired of the ventral surface of the animals using a custom built surface-weighted fluorescence imager with a 670 nm laser for excitation and a 685 nm long-pass emission filter for detection (Pogue, et al. (2004) Technol. Cancer Res. Treat. 3:15-21). Mice were injected with either a surfactant-solubilized free PPa solution in an excipient mixture of 2% ethanol/1% TWEEN 80/phosphate-buffered saline (PBS); a partially PEG2(20 kDa)ylated (˜63 molar %) sPPp conjugate formulation in PBS; or a sPPp-ERBITUX® conjugate formulation (˜11.5 sPPp/monoclonal antibody with ˜42 molar % sPPp noncovalently bound) in PBS. The free PPa solution in 2% ethanol/1% TWEEN 80/PBS served as a gold standard control since many injectable photosensitizer-solubilizer preparations of similar composition, especially those of a variety of PPa derivatives, have frequently been used in the past for both photodiagnostic and phototherapeutic applications (Gurfinkel, et al. (2000) Photochem. Photobiol. 72:94-102; Dougherty, et al. (2002) Photochem. Photobiol. 76:91-7; Bellnier, et al. (2003) supra).

Several striking qualitative observations were drawn from the imaging experiments. First, it was observed that the surfactant-solubilized free PPa solution cleared very rapidly from the circulation via the hepatobiliary system, whereas the sPPp conjugate formulations circulated for much longer and were cleared at much slower rates via both the hepatobiliary and renal/urinary systems. These results indicate that the unique configurations of PEG in the sPPp conjugate formulations dramatically protected the PPa chromophore component from being rapidly captured and cleared by the liver, which was not the case for the gold standard surfactant-solubilized free PPa solution. Second, tumor contrast in the mice injected with the sPPp conjugate formulations was substantially more prominent, longer-lived, and brighter than in the mouse injected with the surfactant-solubilized free PPa solution. This was likely related to the fact that the sPPp conjugate formulations circulated for much longer than the surfactant-solubilized free PPa solution, and longer circulation times translated to improved passive tumor targeting as a result of the generalized EPR effect of solid tumors (Matsumura & Maeda (1986) supra). Finally, it was evident that the actively targeted sPPp-ERBITUX® conjugate formulation exhibited complex pharmacokinetics and an extremely prolonged retention time. The complex pharmacokinetics stems partly from the initial strong fluorescence quenching of the sPPp-ERBITUX® conjugate, which then undergoes dequenching as it degrades within the body. The extremely prolonged retention of the sPPp-ERBITUX® conjugate, particularly in the tumor, indicates that the conjugate was being preferentially taken up and sequestered via receptor-mediated endocytosis by the EGFR overexpressing A-431 tumor cells; i.e., the conjugate was actively targeting the EGFR in the intended manner.

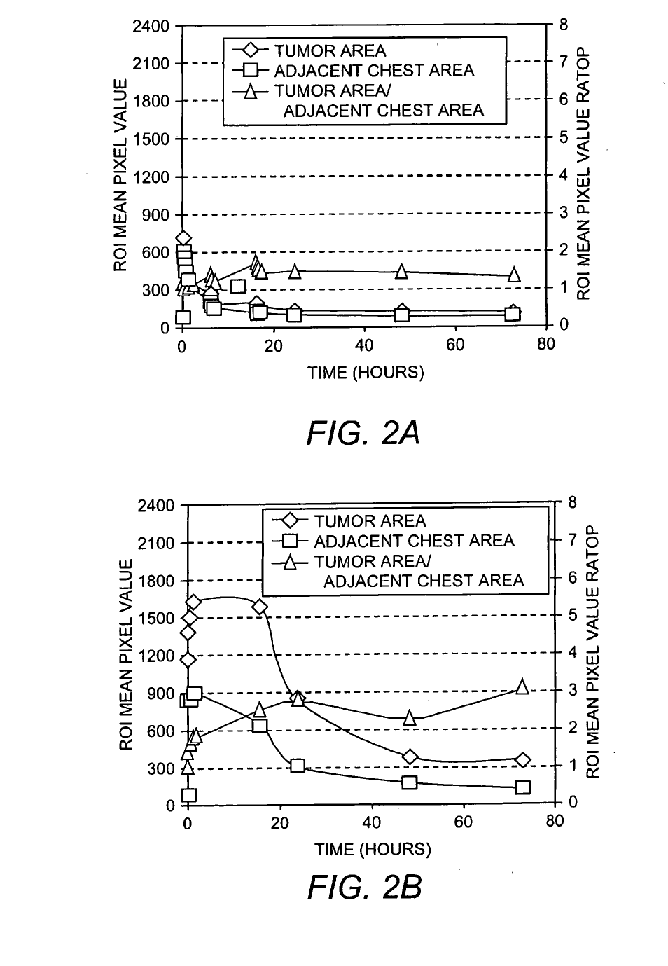

As a quantitative measure of the tumor contrast generated by the surfactant-solubilized free PPa solution and the different sPPp conjugate formulations, region of interest (ROI) mean pixel values of the tumor area and control adjacent chest area were measured from the imaging data sets. FIGS. 2A-2C show mean pixel value time-course plots of the tumor area and control adjacent chest area ROIs as well as the ratio of these quantities. The results of this analysis indicated that the surfactant-solubilized free PPa solution generated relatively weak tumor contrast with a peak tumor area to adjacent chest area mean pixel value ratio of only ˜1.5 at ˜16 hours post-injection (see FIG. 2A). In comparison, the partially PEG2(20 kDa)ylated sPPp conjugate formulation generated tumor contrast of ˜2.5-3 for times >16 hours post-injection (see FIG. 2B), and the sPPp-ERBITUX® conjugate formulation generated tumor contrast of ˜3.75-7.4 for times>24 hours post-injection (see FIG. 2C). These quantitative observations definitively show that the sPPp conjugate formulations are superior tumor contrast agents for photodiagnostic applications compared to the gold standard surfactant-solubilized free PPa solution.

Another key feature of the time-course ROI plots in FIG. 2 is the overall brightness of the tumor fluorescence at the time of peak tumor contrast. For the surfactant-solubilized free PPa solution, the tumor fluorescence was rapidly decreasing at roughly an exponential rate immediately following injection, and at the time of peak tumor contrast, the tumor fluorescence was already approaching pre-injection fluorescence levels (see FIG. 2A). This means that relatively little free PPa remained in the tumor at the time of best tumor contrast. By comparison, the sPPp conjugate formulations exhibited much brighter tumor fluorescence during corresponding times of peak tumor contrast (see FIGS. 2B and 2C). These results indicate that the sPPp conjugate formulations allow high phototherapeutic ratios, and consequently, they permit much safer and more effective phototherapeutic treatments than the gold standard surfactant-solubilized free PPa solution.

### Example 3

### Phototherapy with Passively Targeted PEGylated Peptide-Chromophore Conjugate

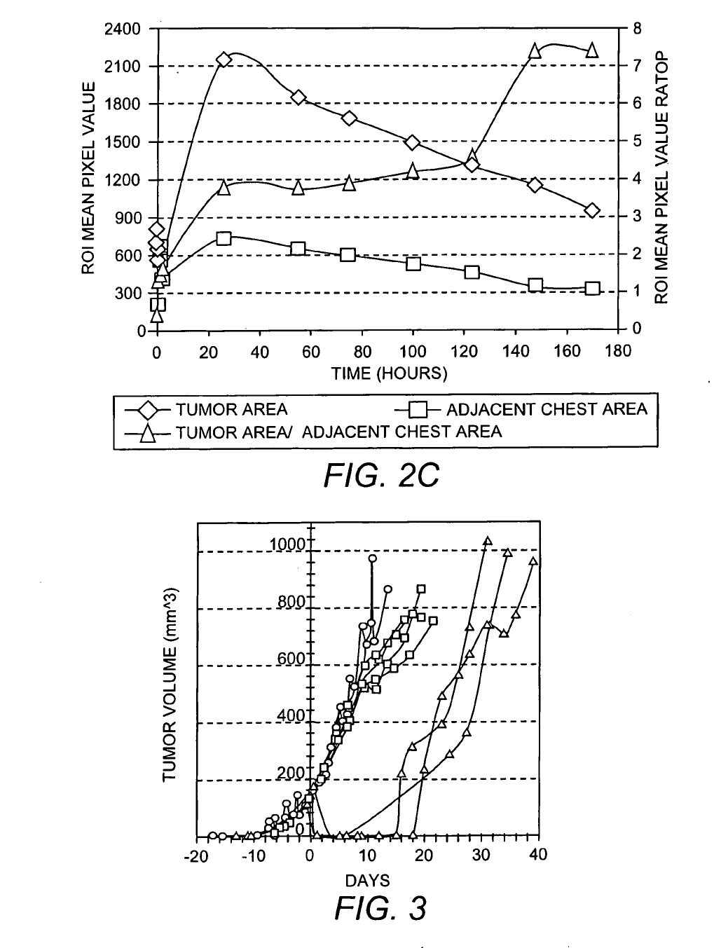

Preliminary tumor growth delay studies comparing the in vivo phototherapeutic effects of the partially PEG2(20 kDa)ylated sPPp conjugate to the gold standard surfactant-solubilized free PPa solution are shown in FIG. 3. A-431 human tumor xenografts were grown in mice as previously described, and when tumors reached ˜150 mm³, mice were injected via the tail vein or retro-orbitally with 60 to 80 nmoles PPa content in a bolus volume of approximately 200 microliters. Sixteen hours post-injection, the entire tumor surface along with a 1-2 mm margin of skin around the tumor edge was uniformly irradiated with the expanded beam of a 670 nm laser at an incident fluence rate of ˜50 mW/cm². Six mice that received no injections and no irradiations served as a control group. Tumor growth delays resulting from the tumoricidal effects of the phototherapeutic treatments were determined with respect to the control group. For control mice, it took roughly 8.1 days for tumors to quadruple in size from 150 mm³ to 600 mm³, and for mice treated with 60 nmoles surfactant-solubilized free PPa and 100 J/cm² light, it took only slightly longer, 12.6±2.4 days (i.e., a tumor growth delay of ˜4.5 days compared to the control group). However, for mice treated with 60 nmoles of partially PEG2(20 kDa)ylated sPPp conjugate and just 50 J/cm² light, tumors took substantially longer to quadruple in size, 27.8±2.0 days (i.e., a tumor growth delay of ˜19.7 days). Even more impressive, for one mouse treated with 80 nmoles of partially PEG2(20 kDa)ylated sPPp conjugate and 100 J/cm² light, the tumor growth delay was ˜66 days. Although such a treatment regimen must have been very close to the curative dose required to completely eliminate all vestiges of the tumor, such treatment is generally too taxing and can be lethal, most likely due to excessive edema and nonspecific damage to sensitive normal tissues. Apart from the potential risks of overtreatment, the above results indicate that sPPp conjugate formulations have distinct advantages over the gold standard surfactant-solubilized free PPa solution. The sPPp conjugates provided superior tumor contrast and at the same time also allowed highly effective phototherapeutic treatments. In contrast, the surfactant-solubilized free PPa solution only provided weak tumor contrast after most of the PPa content had already cleared from the body; consequently, in this case, simultaneously locating and treating a tumor would have been infeasible. In fact, the surfactant-solubilized free PPa solution was most phototherapeutically active when irradiations were performed immediately after injection (e.g., within 5 to 15 minutes post-injection) during a period when there was little or no tumor contrast but there was still a significant amount of photoactive PPa chromophore in circulation. However, since the surfactant-solubilized free PPa solution cleared so quickly into the liver, controlling the phototherapeutic dosage would be challenging given the restricted irradiation time-window. Furthermore, when irradiations were performed immediately after injection, the free PPa solution was still predominantly in the bloodstream, and a very high incidence of lethality was observed, presumably due to damage of crucial major vessels.

### Example 4

### Phototherapy with Passively or Actively Targeted PEGylated Peptide-Chromophore Conjugate

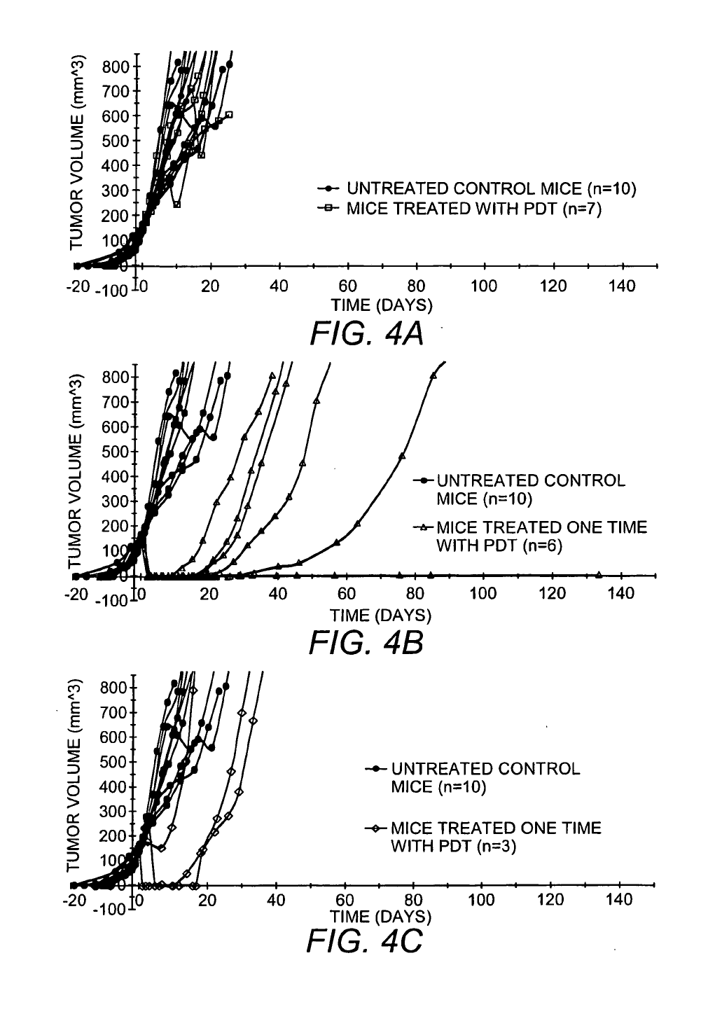

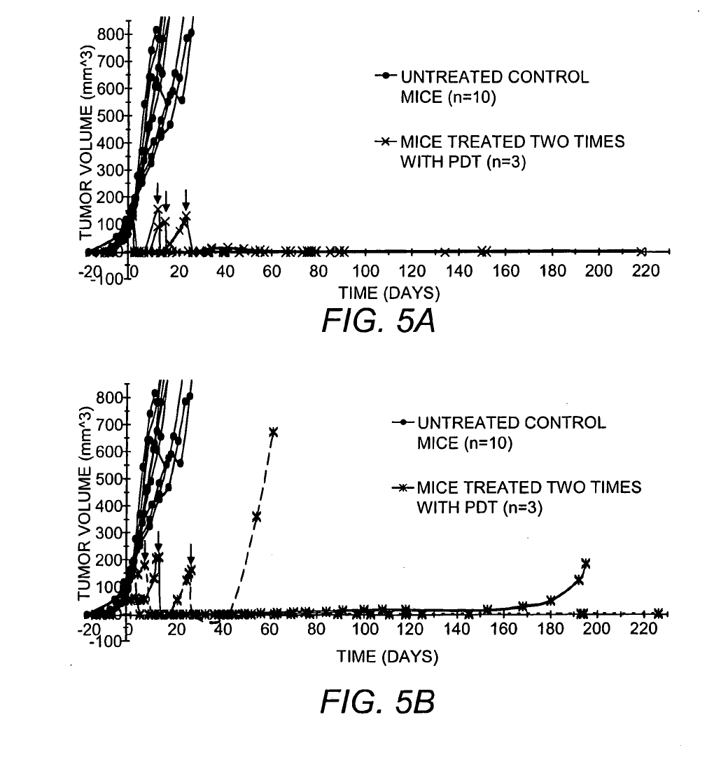

Additional tumor growth delay photodynamic therapy (PDT) studies were conducted demonstrating the advantages of using passively or actively targeted PEGylated peptide-chromophore conjugates over conventional surfactant-solubilized photosensitizer formulations. The results of these analyses are shown in FIGS. 4 and 5. As shown in FIG. 4A, PDT with a surfactant-solubilized PPa formulation resulted in no significant delay in tumor growth compared to untreated control mice that received no photosensitizer and no light (the data for the untreated control mice and the mice that were PDT treated with surfactant-solubilized PPa were largely overlapping each other). In contrast, PDT treatment with an Ac-sPPp formulation yielded substantial delays in tumor growth compared to the untreated control mice, and one mouse remained tumor-free out to at least ˜130 days post-PDT (FIG. 4B). Similarly, PDT treatment with the actively targeted ERBITUX-sPPp immunoconjugate also gave notable delays in tumor growth compared to the untreated control mice (FIG. 4C).

To achieve more prolonged tumor growth delays or tumor elimination, repeat-PDT treatment regimens were investigated (FIG. 5). FIG. 5A shows that two PDT treatments with the Ac-sPPp essentially resulted in tumor elimination (mice remained tumor-free out to >130 days post-PDT and in one case, the mouse remained tumor-free out to 220 days post-PDT). FIG. 5B shows that two PDT treatments with the ERBITUX-sPPp immunoconjugate was much more effective than a single PDT treatment, and one mouse remained tumor-free out to >220 days post-PDT. While the two PDT treatments with the ERBITUX-sPPp immunoconjugate were not as effective as two PDT treatments with the Ac-sPPp formulation, this have been partly related to the length of the time delay between the two PDT treatments. As seen in FIG. 5B, the mouse that had the shortest time delay between the first and second PDT treatments with ERBITUX-sPPp (depicted by the short-dashed line curve) had the most effective tumor delay response, while the mouse that had the longest time delay (depicted by the long-dashed line curve) had the least effective tumor delay response.

Control experiments were also performed, which included injecting mice with the sPPp formulations but not exposing them to light. In all these control experiments, there were no significant “dark effects” of the sPPp formulations; i.e., there were no noticeable delays in tumor growth compared to the untreated controls shown in FIGS. 4 and 5.

These data clearly demonstrate that the PEGylated peptide-chromophore conjugate formulations of the present invention are much more effective PDT agents than conventional surfactant-solubilized photosensitizer formulations.

### Example 5

### Additional Applications

In experiments disclosed herein, it was observed that the sPPp-ERBITUX® conjugate formulation successfully targeted and photodynamically killed EGFR-overpressing target cells while largely sparing non- or low-expressing EGFR nontarget cells. Thus, it is contemplated that the sPPp-ERBITUX® conjugate can be used phototherapeutically effective in an in vivo setting. Moreover, it is contemplated that other types of actively targeted sPPp-anti-cancer monoclonal antibody conjugate formulations (e.g., conjugates made from the anti-HER2 MAb, Herceptin, an antibody that is now widely used in the clinic to treat aggressive breast cancers) can be produced. In addition, the use of both passively and actively targeted conjugate formulations in multi-step photodetection/phototherapy regimens are contemplated. One approach is to inject a small or moderate dose of passively or actively targeted conjugate followed by a prolonged incubation period (e.g., 3 to 72 hours) in order to locate a tumor or other type of vascular lesion, and once sufficient lesion contrast has developed allowing the lesion margins to be identified, inject a second phototherapeutically-effective dose of passively targeted conjugate followed by immediate irradiation (e.g., within 5 to 30 minutes after the second injection) of the demarcated lesion area, which should mainly destroy the abnormal lesion vasculature. It is believed that such an approach will provide more precise targeting and better protect normal surrounding tissues because the lesion area can first be demarcated permitting more accurate aiming of the phototherapeutic excitation light; and the procedure takes advantage of early irradiation procedures which seem to avoid excessive edema and undue damage to adjacent normal tissues, since the photoactive compound remains predominantly in the vessels and does not have time to extravasate significantly into the interstitium where it can come into contact with cells of normal tissues (see U.S. Pat. Nos. 5,770,619 and 6,058,937). One caveat of this approach is that extra precaution will have to be exercised to avoid irradiating any nearby large normal vessels, which if substantially damaged, could result in unacceptable morbidity or even death.

Hybrid conjugate formulations are also contemplated for multimodality imaging applications and therapies. For instance, the PEGylated peptide-chromophore conjugate compositions could be co-labeled with radioisotopes for nuclear imaging, which could then be used to complement photodiagnostic imaging techniques and phototherapy. The advantage of nuclear imaging is that there are no limitations imposed by the depth of tissue, whereas photodiagnostic methods and phototherapy are normally restricted to shallow tissue depths due to the limited tissue penetration of visible and near infrared light. A combination chromophore/radioisotope hybrid conjugate could overcome these limitations by allowing lesions both deep and shallow in tissue to be detected and imaged optimally. Once lesions have been identified and located by combined nuclear and photodetective methods, it would be possible to deliver light for phototherapy to almost anywhere in the body, either superficially or deep in tissues, using fiber optics and other specially designed optical components such as diffusers.

What is claimed is:

1. A PEGylated peptide-chromophore conjugate composition comprising: 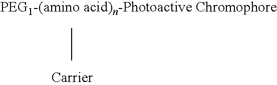
wherein PEG₁ is a linear PEG of 10 to 25 PEG units, n is 1 to 10, and the carrier is branched PEG.

2. The composition of claim 1, wherein one or more of the amino acids contain a function group.

3. The composition of any preceding claim, wherein one or more of the amino acids is lysine.

4. The composition of any preceding claim, wherein n is 2 to 10 and the amino acids are Asp and Lys.

5. The composition of any preceding claim, wherein the photoactive chromophore is selected from the group of chlorin, cyanine, purpurin, porphyrin and pro-photosensitizer.

6. The composition of any preceding claim, wherein the carrier is a passive targeting carrier or an active targeting carrier.

7. The composition of claim 6, wherein the passive targeting carrier is a branched PEG.

8. The composition of claim 7, wherein the branched PEG is double branched with a size of at least 20 kDa.

9. The composition of claim 7 or 8, wherein the PEG₁:(amino acid)ₙ-photoactive chromophore ratio is 1 and the branched PEG:PEG₁-(amino acid)ₙ-photoactive chromophore ratio of <1.

10. The composition of claim 6, wherein the active targeting carrier mimics a ligand that binds preferentially to an overexpressed or overactive oncogenic receptor.

11. The composition of claim 6, wherein the active targeting carrier mimics an enzyme substrate that can be cleaved by a tumor-associated enzyme.

12. The composition of any preceding claim, wherein n is 2 to 10 and wherein at least one amino acid is conjugated to a branched PEG and one amino acid is conjugated to an active targeting carrier.

13. A micellular formulation comprising the composition of any preceding claim.

14. A method for synthesizing the PEGylated peptide-chromophore conjugate of claim 1 comprising
a) linking a linear PEG of 10 to 25 PEG units to an amino acid-photoactive chromophore conjugate via solid phase synthesis, and
b) attaching a branched PEG to an amino acid side chain of the product of a), thereby producing a PEGylated peptide-chromophore conjugate.

15. A micellular formulation comprising at least one molecule of:
PEG₁-(amino acid)ₙ-Photoactive Chromophore noncovalently associated with one molecule of: 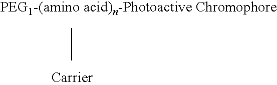
wherein PEG₁ is a linear PEG of 10 to 25 PEG units, and n is 1 to 10.

16. The composition of claim 15, wherein one or more of the amino acids contain a function group.

17. The composition of claim 15 or 16, wherein one or more of the amino acids is lysine.

18. The composition of any one of claims 15 to 17, wherein n is 2 to 10 and the amino acids are Asp and Lys.

19. The composition of any one of claims 15 to 18, wherein the photoactive chromophore is selected from the group of chlorin, cyanine, purpurin, porphyrin and pro-photosensitizer.

20. The composition of any one of claims 15 to 19, wherein the carrier is a passive targeting carrier or an active targeting carrier.

21. The composition of claim 20, wherein the passive targeting carrier is a branched PEG.

22. The composition of claim 21, wherein the branched PEG is double branched with a size of at least 20 kDa.

23. The composition of claim 21 or 22, wherein the PEG₁:(amino acid)ₙ-photoactive chromophore ratio is 1 and the branched PEG:PEG₁-(amino acid)ₙ-photoactive chromophore ratio of <1.

24. The composition of claim 20, wherein the active targeting carrier mimics a ligand that binds preferentially to an overexpressed or overactive oncogenic receptor.

25. The composition of claim 20, wherein the active targeting carrier mimics an enzyme substrate that can be cleaved by a tumor-associated enzyme.

26. The micellular formulation of any one of claims 15 to 25, wherein n is 2 to 10 and wherein at least one amino acid is conjugated to a passive targeting carrier and one amino acid is conjugated to an active targeting carrier.

27. A method for producing the micellular formulation of claim 15 comprising
a) linking a linear PEG of 10 to 25 PEG units to an amino acid-photoactive chromophore conjugate via solid phase synthesis, and
b) attaching one or more carriers to amino acid side chains of the product of a), and
c) noncovalently associating at least one molecule of the product of a) with one molecule of the product of b), thereby producing a micellular PEGylated peptide-chromophore-carrier conjugate formulation.

28. A method for diagnosing a lesion comprising administering to a subject having or suspected of having a lesion a composition of claim 13 or 15 and imaging the photoactive chromophore, thereby diagnosing the lesion in the subject.

29. A method for treating a lesion comprising administering to a subject with a lesion a composition of claim 13 or 15 and a suitable light dose thereby treating the lesion.

30. The method of claim 29, wherein the lesion is cancer.
